(19) United States
(12) Patent Application Publication
(10) Pub. No.: US 2010/0318101 A1
(43) Pub. Date: Dec. 16, 2010

(54) COUPLING STRUCTURE OF SURGICAL INSTRUMENT

(76) Inventor: Seung Wook CHOI, Seoul (KR)

Correspondence Address: BIRCH STEWART KOLASCH & BIRCH, PO BOX 747, FALLS CHURCH, VA, 22040-0747, US

(21) Appl. No.: 12/867,544

(22) PCT Filed: Oct. 7, 2008
(86) PCT No.: PCT/KR08/05874

§ 371 Date: Aug. 13, 2010

(30) Foreign Application Priority Data
Feb. 15, 2008 (KR) 10-2008-0013970

(51) Int. Cl. A61B 19/00

(52) U.S. Cl. 606/130

(57) ABSTRACT

Disclosed is a coupling structure of surgical instrument. The coupling structure for a surgical instrument comprises a housing and a plurality of disc shaped driving wheels disposed in the housing, wherein the driving wheels are stacked along a driving axis that passes through the driving wheels, and are supplied with driving power from a plurality of actuators that are disposed to correspond respectively to the plurality of driving wheels, and provides a light compact surgical robot by disposing driving wheels in a piling arrangement, which also allows an used instrument to be automatically replaced with a new one.

### CROSS-REFERENCE TO RELATED APPLICATIONS

This application is the National Phase of PCT/KR2008/005874 filed on Oct. 7, 2008, which claims priority under 35 U.S.C. 119(a) to Patent Application No. 10-2008-0013970 filed in the Republic of Korea on Feb. 15, 2008, all of which are hereby expressly incorporated by reference into the present application.

### BACKGROUND

The present invention relates to a coupling structure of a surgical instrument.

Surgery refers to a medical specialty that uses operative manual and instrumental techniques on the tissues of a patient to treat a pathological condition. Surgical robots have been proposed as an alternative for performing an excision surgery, which needs cutting tissues to treat or remove the organ within the body, to reduce blood loss, pain and improve precision.

The surgical robot consists of a master robot generating and transmitting signals according to a manipulation of a surgeon and a slave robot applying the manipulation directly to the patient according to the signals from the master robot. The master robot may be integrated with the slave robot or may be separated from the slave robot.

The slave robot comprises robotic arms for surgical manipulation, and at a fore end of the robot arm is formed an instrument. The existing instrument 54 comprises, as shown in FIG. 1, a housing 108, a shaft 102 extending from the housing 108, and a pincer shaped manipulation part 112 formed at an end of the shaft 102 and inserted into a surgical site. An interface part 110 is formed at a bottom side of the housing 108.

As shown in FIG. 2, a plurality of wheel shape driving elements 118 are combined at the bottom side of the existing instrument 54. The driving elements 118 are wound with wires connected with the manipulation part 112, so that tension on the wires generated by the revolution of the driving elements 118 causes the manipulation part 112 to operate.

In order to mount the instrument 54 on the robotic arm, an adaptor 128, as shown in FIG. 3, is combined with the fore-end of the robotic arm. The adaptor 128 is formed with a guide wing and actuators. The interface part 110 of the housing 108 is coupled with the adaptor 128 through the guide wing and the actuator has a shape corresponding with the driving element to provide revolution power to the driving element.

As described above, the existing instrument 54 has a coupling structure in which the instrument 54 is combined with the robotic arm through the adaptor 128, and performs surgery by operating the manipulation part 112 by revolving the driving element 118 through the actuator formed in the adaptor 128.

However, in such a coupling structure, there is a limit to reducing the size of the housing because the driving elements should be disposed on the bottom surface of the housing. As seen in FIG. 2, when two arrays of the driving elements are disposed, the bottom plane should be twice as wide as the diameter of the driving elements.

This limit in reducing the size of the instrument becomes an obstacle to miniaturizing the surgical robot and also to applying a technology for automatically replacing the disposable instrument.

### SUMMARY

The present invention aims to provide a coupling structure of a surgical instrument that can miniaturize a surgical robot by minimizing the size of the surgical instrument, and that can serve as a technology for enabling automatic replacement of the disposable instrument.

According to one aspect of the present invention, a coupling structure for a surgical instrument is provided, the coupling structure comprising a housing and a plurality of disc shaped driving wheels disposed in the housing, wherein the driving wheels are stacked along a driving axis that passes through the driving wheels, and are supplied with driving power from a plurality of actuators that are disposed to correspond respectively to the plurality of driving wheels.

The coupling structure may further comprise a plurality of wires that are respectively wound around the driving wheels and deliver driving power to a manipulation part formed on a fore-end of the surgical instrument.

The driving axis may be perpendicular to a surface the driving wheel.

The driving axis may pass through a center of the driving wheel.

A thickness of the housing may amount approximately to a sum of thickness of the plurality of driving wheels.

The plurality of actuators may be stacked along an axis, and each actuator may comprise a wheel that is engaged with a corresponding driving wheel by a rolling contact.

A circumferential surface of the driving wheel may comprise rubber material on which a plurality of protrusions are formed.

A surface of the driving wheel may be exposed to a outer surface of the housing, and each actuator may comprise a wheel contacting a corresponding disc of the driving wheel.

A surface of the driving wheel facing the actuator may comprise rubber material on which a plurality of protrusions are formed.

A gear may be formed on the surface of the driving wheel facing the actuator, and the actuator may comprise a driving gear that forms a gear combination with the driving wheel.

The coupling structure may further comprise a plurality of sub wheels that are disposed in the housing to correspond respectively to the plurality of driving wheels and respectively form a pulley combination with the driving wheels, wherein each actuator comprises a slider that applies a tension to the pulley by a slide movement.

A gear may be formed on a circumferential surface of the driving wheel and each actuator may comprise a driving gear forming a gear combination with the driving wheel.

The coupling structure may further comprise a plurality of sub wheels that are exposed on a side of the housing to correspond respectively to the plurality of driving wheels, and form a gear combination or a pulley combination with the driving wheels, wherein each actuator comprises a driver applying torque to corresponding sub wheel.

On an exposed area of the sub wheel may be formed a groove, and on an end of the driver may be formed a protrusion having a shape corresponding to the groove.

Additional aspects, features, and advantages will be elucidated from the following drawings, claims, and specification.

This invention provides a light compact surgical robot by disposing driving wheels in a piling arrangement, which also allows an used instrument to be automatically replaced with a new one.

### BRIEF DESCRIPTION OF THE DRAWINGS

FIGS. 1 through 3 illustrate a surgical instrument according to prior art.

FIG. 4 is a perspective view illustrating a coupling structure of a surgical instrument according to an embodiment of the present invention.

FIG. 5 is a side view of a coupling structure of a surgical instrument according to an embodiment of the present invention.

FIG. 6 is a side view illustrating a coupling structure of a surgical instrument according to another embodiment of the present invention.

FIG. 7 is a side view illustrating a coupling structure of a surgical instrument according to another embodiment of the present invention.

FIG. 8 is a perspective view illustrating a coupling structure of a surgical instrument according to another embodiment of the present invention.

FIG. 9 is a perspective view illustrating a coupling structure of a surgical instrument according to another embodiment of the present invention.

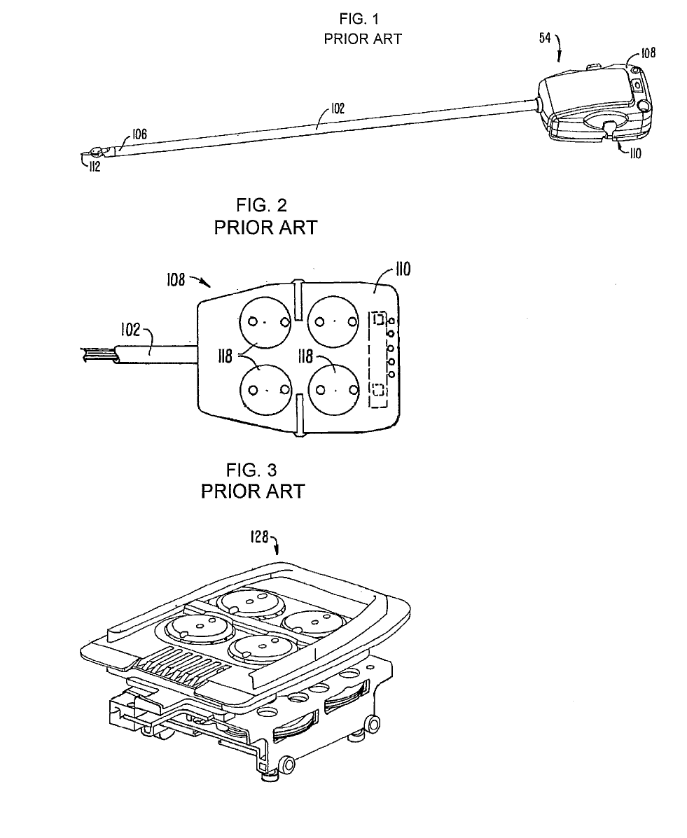

### DETAILED DESCRIPTION

Although a few embodiments of the present invention have been shown and described, it will be appreciated by those skilled in the art that changes may be made in these embodiments without departing from the principles and spirit of the invention, the scope of which is defined in the appended claims and their equivalents. Also, specific descriptions on related prior art will be omitted in order to concentrate on the gist of the present invention.

The terms first, second, third and the like in the description and in the claims, are used for distinguishing between similar elements and not necessarily for describing a sequential or chronological order.

The present invention will be described with respect to particular embodiments and with reference to certain drawings but the invention is not limited thereto and is limited only by the claims. Where an indefinite or definite article is used when referring to a singular noun e.g. “a” or “an”, “the”, this includes a plural of that noun unless something else is specifically stated.

It is to be noted that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof.

Hereinafter, same reference numerals refer to the same or similar parts throughout the drawings and repetitive descriptions about the same element are omitted.

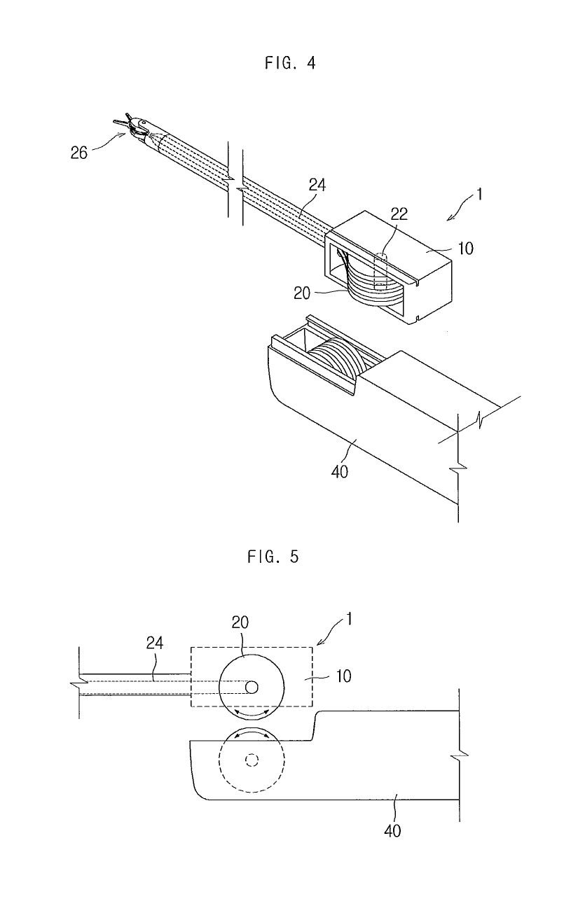

FIG. 4 illustrates in a perspective view a coupling structure of a surgical instrument according to an embodiment of the present invention. In FIG. 4 are shown an instrument 1, a housing 10, a driving wheel 20, a driving axis 22, a wire 24, a manipulation part 26, and an actuator 40.

A feature of this embodiment is that a width of the housing 10 of the instrument 1 can be minimized by stacking the driving wheels 20 of the instrument 1 along an axis direction.

The instrument 1 comprises the housing 10, a shaft extending from the housing 10, and the manipulation part 26 combined with an end of the shaft. In the housing 10, the driving wheels 20 are disposed not in a planar arrangement but in a stacked arrangement.

A width of the housing 10 can be narrowed by stacking the disc shaped driving wheels 20 in the direction of an axis penetrating the driving wheels 20, instead of disposing the driving wheels 20 in a planar arrangement. For example, the prior instrument in FIG. 2 should be at least twice as wide as the diameter of the driving wheel However, when the driving wheels 20 are stacked as shown in FIG. 4, the thickness of the housing 10 corresponds to the height of the stack of the driving wheels 20, and the width of the housing 10 can be narrowed to correspond to the diameter of the driving wheels 20.

As described above, the present invention can contribute to reducing the size and weight of a surgical robot by reducing the size of the housing 10 of the surgical instrument 1. In particular, a plurality of instruments 1 can be supplied sequentially in a cartridge type, thereby facilitating the introduction of an automatic replacement system for used instruments 1.

The disc type driving wheel 20 of the instrument 1 revolves around the driving axis 22, which perpendicularly penetrates a center of the discs. Accordingly, the driving wheels 20 may be stacked in a direction of the driving axis 22. The width of the housing 10 can be minimized when stacking the driving wheels 20 in the direction of the axis 20, and the thickness of the housing 10 can be minimized when the driving axis 22 is perpendicular to the driving wheels 20.

However, the axis 22 does not necessarily have to be perpendicular to the wheels 20, and the wheels 20 may be disposed, for example, in a zigzag arrangement.

The instrument 1 in which the driving wheels 20 are repeatedly arrayed as shown in FIG. 4 is disposed in a predetermined position of the robot arm. Similar to an existing instrument, the housing 10 of the present embodiment may have an interface part on its bottom side, and a guide wing may be formed on a corresponding position of the robot arm that allows the interface part to be fixed. Details on the interface part and the guide wing will not be described.

When disposed in the predetermined position of the robot arm, the instrument 1 is provided with driving power from the robot arm. Each wheel 20 is wound with the wire 24, which is connected through the shaft with the manipulation part 26. Accordingly, the driving wheels 20 revolve due to the driving power from the robot arm, generating tension on the wire 24, which causes units of the manipulation part 26 to operate.

Hereinafter, a unit in the robot arm delivering driving power to the instrument 1 will be referred to as an actuator. The actuator 40 may comprise a wheel, a slider, a gear, and the like as a means for delivering driving power to each driving wheel 20. The actuator 40 will be described in detail with reference to FIGS. 5 through 7.

FIG. 5 is a side view showing a coupling structure of a surgical instrument according to an embodiment of the present invention. In FIG. 5 are illustrated an instrument 1, a housing 10, a driving wheel 20, a wire 24, and an actuator 40.

A feature of this embodiment is that the actuator 40 comprises a plurality of wheels, each wheel being engaged with a corresponding driving wheel 20 by rolling contact. The wheels contact the driving wheels 20 such that when the wheel in the actuator 40 rotates, the engaged driving wheel 20 also rotates in synchronization therewith.

In this way, driving power can be provided through the actuator 40, and the accuracy of the manipulation can be adjusted by altering the ratio of the radius of the driving wheel 20 to the radius of the wheel of the actuator 40. More specifically, when the wheel of the actuator 40 is larger than the driving wheel 20, a small amount of rotation of the wheel of the actuator 40 allows the driving wheel a relatively larger amount of rotation. Conversely, when the driving wheel 20 is larger than the wheel of the actuator 40, the driving wheel performs a smaller amount of rotation than the wheel of the actuator 40. Therefore, the radius ratio will be determined depending on a desired accuracy of an operation.

It is recommendable that the friction coefficient of a circumferential surface of the wheel of the actuator 40 and/or of the driving wheel 20 be high in order to enhance the efficiency in delivering the driving power. For example, a plurality of protrusions (H may be formed on the circumferential surface, or the circumferential surface may be made of a material having a high frictional coefficient such as rubber, so that the rotational power of the wheel of the actuator 40 can be delivered to the driving wheel efficiently.

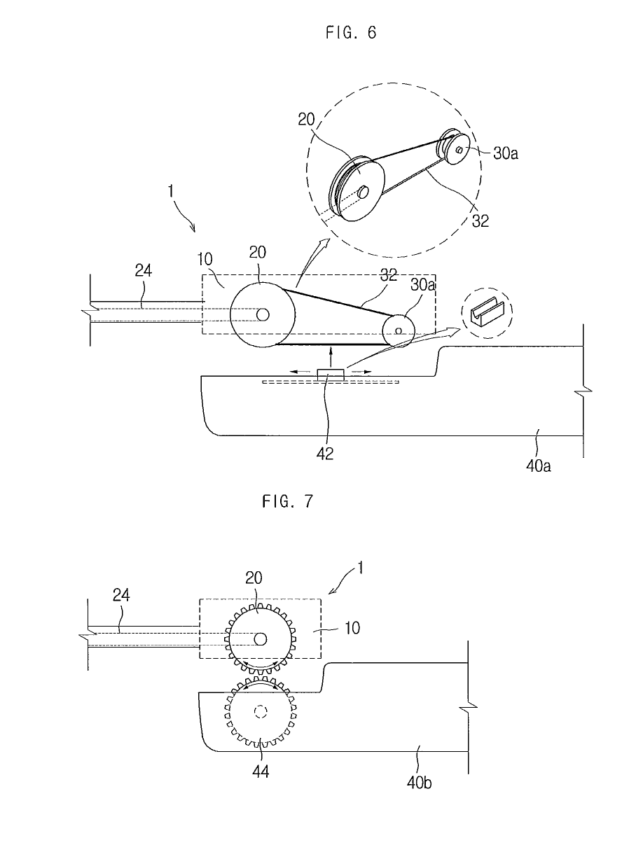

FIG. 6 is a perspective view illustrating a coupling structure of a surgical instrument according to another embodiment of the present invention. In FIG. 6 are shown an instrument 1, a housing 10, a driving wheel 20, a wire 24, a sub wheel 30a, a pulley 32, an actuator 40a, and a slider 42.

A feature of this embodiment is that the sub wheel 30a is additionally disposed in the housing 10 to be connected with the driving wheel 20 by the pulley 32, and a plurality of sliders 42 are disposed in the actuator 40a to apply tension to the pulley 32.

As shown in FIG. 6, when the pulley 32 is pulled toward the driving wheel 20 or the sub wheel 30a, the driving wheel 20 accordingly rotates clockwise or counter-clockwise.

By comprising the slider 42 as a means for applying tension to the pulley 32, the actuator 40a provides driving power to the instrument 1 as in the preceding embodiment of FIG. 5. The slider 42 corresponding to the driving wheel 20 moves in a reciprocating motion, pulling the pulley 32 toward the driving wheel 20 or the sub wheel 30a, thereby rotating the driving wheel 20 in synchronization therewith.

The manipulation part 26 of the instrument 1 moves within a predetermined range, which means that the rotation of the driving wheel 20 should be restricted within a predetermined range. In the preceding embodiment in FIG. 5, the rotation of the wheel of the actuator 40a may be restricted in order to restrict the rotation of the driving wheel 20, and to this end, brake elements may be formed on certain positions of the wheel.

In the present embodiment, a moving guide of the slider 42 may be designed to have a length that allows the slider 42 to move within a restricted range, thereby putting a limit on the movement of the manipulation part 26 of the instrument 1.

It is recommendable that the friction coefficient between the slider 42 and the pulley 32 be high, in a similar manner to the preceding embodiment in FIG. 5. A groove ( [symbol] ) may be formed on the slider 42 so that the pulley 32 can be inserted in the slider 42, and the surface of the slider 42 and/or pulley 32 may be made of a material with a high frictional coefficient such as rubber, so that the movement of the slider 42 can be converted into the rotation of the driving wheel 20 efficiently.

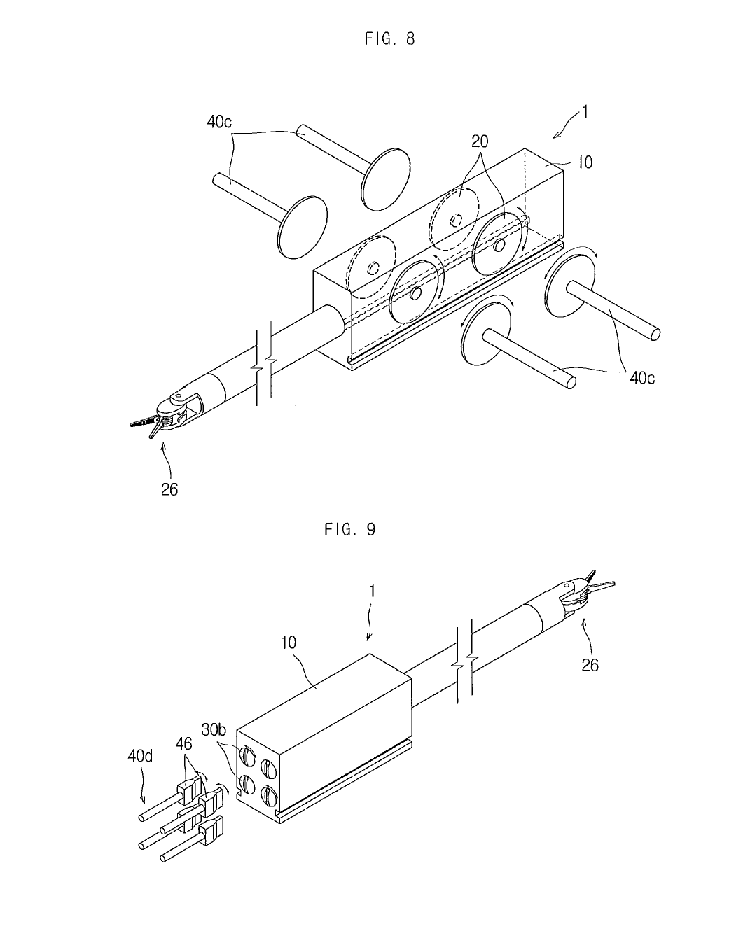

FIG. 7 is a side view illustrating a coupling structure of a surgical instrument according to another embodiment of the present invention. In FIG. 7 are shown an instrument 1, a housing 10, a driving wheel 20, a wire 24, an actuator 40b, and a driving gear 44.

A feature of this embodiment is that gears are formed around the circumference of the driving wheels 20, and the actuator 40b comprises the driving gears 44, each forming a gear-combination with a counterpart driving wheel 20, so that the driving wheel 20 rotates in synchronization with the driving gear 44.

The gear combination for the driving gear 44 and the driving wheel 20 may be a spur gear, as shown in FIG. 7, a helical gear, a worm gear, a rack and pinion, or the like.

With such a configuration, the actuator 40b can deliver driving power, and a gear ratio between the driving wheel 20 and the driving gear 44 may be altered to adjust the accuracy of the instrument 1.

Unlike the preceding embodiments in FIG. 5 and FIG. 6, the role of the frictional coefficient is relatively unimportant since the gear is efficient in delivering driving power.

As seen in the above, the present invention provides a surgical instrument in which the width or the size of a housing may be reduced by altering the size of a driving wheel, optimizing the arrangement of driving wheels, and employing sub wheels.

FIG. 8 is a perspective view illustrating a coupling structure of a surgical instrument according to another embodiment of the present invention. In FIG. 8 are shown an instrument 1, a housing 10, driving wheels 20, a manipulation part 26, and an actuator 40c.

This embodiment introduces an example of an arrangement for the driving wheels 20 in which a plurality of driving wheels 20 are arranged in pairs and some of the pairs are disposed in a fore part of the housing 10 and the rest of the pairs are disposed in a rear part, while all of the driving wheels are stacked along the driving axis in FIG. 4.

The actuator 40c may have a similar configuration to that in FIG. 4, or in the case that the disc of the driving wheel is partially or entirely exposed at the outside of the housing 10 as shown in FIG. 8, the actuator 40c may have a plurality of wheels clutched with the driving wheels 20 from the outer side of the housing 10. In other words, the actuator 40c has wheels corresponding to the driving wheels 20, thereby rotating the driving wheels 20 in synchronization therewith.

In order for an efficient combination of the wheels of the actuator 40c with the driving wheels 20, the surfaces of the discs of the driving wheels 20 and/or the surfaces of the wheels of the actuator 40c facing the driving wheels 20 may be made of rubber, and protrusions may also be formed on the surfaces. Alternatively, a gear may be formed on the disc of each of the driving wheels 20, and each wheel of the actuator 40c may be a driving gear (not shown) that forms a gear combination with the gear of the driving wheel 20.

The thickness of the housing 10 may be narrower than that in the embodiment shown in FIG. 4 when the driving wheels 20 are aligned in pairs in a lengthwise direction of the housing 10.

FIG. 9 is a perspective view illustrating a coupling structure of a surgical instrument according to another embodiment of the present invention. In FIG. 9 are shown an instrument 1, a housing 10, sub wheels 30b, a manipulation part 26, an actuator 40d, and a driver 46.

Reducing the size of the housing 10 of the instrument 1 as shown in FIG. 8 may facilitate the implementation of an automatic replacement system that supplies a plurality of instruments 1 sequentially in a cartridge type.

In order to apply a replacement system to the instrument 1, one end of each of the sub wheels 30b may be exposed as shown in FIG. 9 at a rear side of the housing 10. The above description on the driving wheels is equally valid for this embodiment.

In the embodiment shown in FIG. 9, the driving wheels in the housing 10 are combined with the sub wheels 30b through gears, wires, pulleys, or the like, and one end of each of the sub wheels 30b is exposed at the rear side of the housing 10. A worm gear combination, for example, may be employed for the combination of the sub wheel 30b and the driving wheel in order to expose one end of the sub wheel 30b at an outer surface of the housing 10.

In the exposed surface of the a sub wheel 30b, there may be formed a slot having a shape of −, +, or the like, as on the head of a screw, and a driver 46 having a tip that is shaped to correspond to the slot may be engaged with the sub wheel 30b, so that driving power can be supplied to the instrument 1.

To be more specific, the driver 46 rotates, causing the sub wheel 30b to rotate, and in synchronization therewith the driving wheel (not shown) that is combined with the sub wheel 30b also rotates, eventually allowing the manipulation part 26 to operate.

Although the present invention is described by referring to certain preferred embodiments, it will be appreciated by those skilled in the art that changes may be made without departing from the principles and spirit of the invention, the scope of which is defined in the appended claims and their equivalents.

1. An instrument for robotic surgery configured to combine with an actuator formed on a robot aim of a surgical robot to be operated by driving power supplied from the actuator, the instrument comprising:
a housing disposed in an area of the robot arm where the actuator is formed;
a shaft combined with the housing and extended from the housing;
a plurality of disc shaped driving wheels disposed in the housing;
a plurality of wires held inside the shaft, each of the plurality of wires having one end thereof wound around one of the driving wheels, respectively, and having tension applied thereto by a rotation of the driving wheel; and
a manipulation part combined with an end of the shaft and combined with the other ends of the wires, the manipulation part operated by driving power supplied from the wires,
wherein the actuator comprises a plurality of disc shaped wheels stacked and rotated along an axis,
the driving wheels are stacked along a driving axis that passes through the disc surface, and respectively engaged with the plurality of wheels of the actuator by rolling contact to be supplied with driving power from the plurality of wheels,
and a thickness of the housing amounts approximately to a sum of thicknesses of the plurality of driving wheels.

2. (canceled)

3. The instrument for robotic surgery of claim 1, wherein the driving axis is perpendicular to a surface the driving wheel.

4. The instrument for robotic surgery of claim 1, wherein the driving axis passes through a center of the driving wheel.

5. The instrument for robotic surgery of claim 1, wherein a gear is formed on a circumferential surface of the driving wheel and each actuator comprises a driving gear forming a gear combination with the driving wheel.

6. (canceled)

7. The instrument for robotic surgery of claim 1, wherein a circumferential surface of the driving wheel comprises rubber material on which a plurality of protrusions are formed.

8. An instrument for robotic surgery configured to combine with an actuator formed on a robot arm of a surgical robot to be operated by driving power supplied from the actuator, the instrument comprising:
a housing disposed in an area of the robot arm where the actuator is formed;
a shaft combined with the housing and extended from the housing;
a plurality of disc shaped driving wheels disposed in the housing;
a plurality of wires held inside the shaft, each of the plurality of wires having one end thereof wound around one of the driving wheels, respectively, and having tension applied thereto by a rotation of the driving wheel; and
a manipulation part combined with an end of the shaft and combined with the other ends of the wires, the manipulation part operated by driving power supplied from the wires,
wherein the actuator comprises a plurality of disc shaped wheels configured to clutch with disc surfaces of the driving wheels, respectively, at both outer sides of the housing,
the driving wheels are stacked along a driving axis that passes through the disc surface and exposed at both outer sides of the housing, the driving wheels configured to clutch the plurality of wheels to be supplied with driving power from the plurality of wheels, and a thickness of the housing amounts approximately to a sum of thicknesses of the plurality of driving wheels.

9. The instrument for robotic surgery of claim 8, wherein a surface of the driving wheel facing the actuator comprises rubber material on which a plurality of protrusions are formed.

10. The instrument for robotic surgery of claim 8, wherein a gear is formed on a surface of the driving wheel facing the actuator, and a driving gear that forms a gear combination with the driving wheel is formed on a surface of the plurality of wheels facing the driving wheel.

11. An instrument for robotic surgery configured to combine with an actuator formed on a robot aim of a surgical robot to be operated by driving power supplied from the actuator, the instrument comprising:
a housing disposed in an area of the robot arm where the actuator is formed;
a shaft combined with the housing and extended from the housing;
a plurality of disc shaped driving wheels disposed in the housing and stacked along a driving axis that passes through the disc surface;
a plurality of sub wheels disposed in the housing to correspond respectively to the plurality of driving wheels and respectively forming a pulley combination with the plurality of driving wheels;
a plurality of wires held inside the shaft, each of the plurality of wires having one end wound around one of the driving wheels, respectively, and having tension applied thereto by a rotation of the driving wheel; and
a manipulation part combined with an end of the shaft and combined with the other ends of the wires, the manipulation part operated by driving power supplied from the wire,
wherein the actuator comprises a plurality of sliders that rotate the driving wheels by applying tension in a direction of movement to the plurality of pulleys which combine the driving wheels with the sub wheels by a slide movement,
and a thickness of the housing amounts approximately to a sum of thicknesses of the plurality of driving wheels.

12. (canceled)

13. An instrument for robotic surgery configured to combine with an actuator formed on a robot arm of a surgical robot to be operated by driving power supplied from the actuator, the instrument comprising:
a housing disposed in an area of the robot arm where the actuator is formed;
a shaft combined with the housing extended from the housing;
a plurality of disc shaped driving wheels disposed in the housing and stacked along a driving axis that passes through the disc surface;
a plurality of sub wheels exposed at the rear side of the housing to correspond respectively to the plurality of driving wheels and forming a gear combination or a pulley combination with the driving wheels;
a plurality of wires held inside the shaft, each of the plurality of wires having one end wound around one of the driving wheels, respectively, and having tension applied thereto by a rotation of the driving wheel; and
a manipulation part combined with an end of the shaft and combined with the other ends of the wires, the manipulation part operated by driving power supplied from the wires,
wherein the actuator comprises a plurality of drivers respectively applying torque to the plurality of sub wheels,
and a thickness of the housing amounts approximately to a sum of thicknesses of the plurality of driving wheels.

14. The instrument for robotic surgery of claim 13, wherein on an exposed area of the sub wheel is formed a groove, and on an end of the driver is formed a protrusion having a shape corresponding to the groove.
